(19) United States
(12) Patent Application Publication
(10) Pub. No.: US 2010/0221205 A1
(43) Pub. Date: Sep. 2, 2010

(54) METHOD OF APPLYING MAKEUP AND A COSMETIC KIT FOR IMPLEMENTING SUCH A METHOD

(75) Inventors: Xavier BLIN, Paris (FR); Coralie CLAUDE-FOLY, Verneuil en Halatte (FR); Sophie BEAUMARD, Villejuif (FR); Pascal ARNAUD, L'Hay les Roses (FR); Francoise BLONDEAU, Choisy le Roi (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/642,746

(22) Filed: Dec. 18, 2009

Related U.S. Application Data
(60) Provisional application No. 61/193,922, filed on Jan. 8, 2009.

(30) Foreign Application Priority Data
Dec. 18, 2008 (FR) 0858767

(51) Int. Cl. A61K 8/19; A61K 8/27; A61K 8/29; A61Q 1/02; A61Q 19/00

(52) U.S. Cl. 424/63; 424/642

(57) ABSTRACT

A method of applying makeup and/or a skincare composition to the skin using a vibrating applicator. The method smoothes the skin and provides microrelief. A cosmetic composition in the form of loose powder is applied to the skin with a vibrating applicator. The cosmetic composition contains a lamellar particulate material present in a proportion by weight that is greater than or equal to 5% relative to the total weight of the composition and hydrocarbon, silicone, and/or fluorinated binder.

### FIELD OF THE DISCLOSURE

The disclosure relates to applying makeup and/or a skincare composition to the skin.

### BACKGROUND

The use of loose powders in particular with respect to complexion makeup is very common.

The function of such powders is to provide color and/or matteness, and to improve the lasting quality of the foundation.

However, their application is often problematic because of their powdery nature. For instance, it is easy to soil clothing during application.

That is why much research has been carried out to find applicators that minimize those drawbacks and make application easier.

Applicators in the form of a powder puff or a sponge do not prevent the powder from blowing about, since the powder does not adhere well, or adheres very little to the applicator.

More recently, paint brushes have been proposed, but the wiping movement of the hand during application, and the lack of adherence of the powder to the bristles of the brush do not enable a completely satisfactory application to be obtained.

### SUMMARY

Thus, there remains a need to develop cosmetic kits and methods of applying makeup and/or skincare compositions that make it possible to avoid the above-mentioned drawbacks.

Exemplary embodiments provide a method of applying makeup and/or a skincare composition to the skin, in particular so as to smooth skin and provide microrelief, the method comprising:
applying a cosmetic skincare composition in the form of loose powder using a vibrating applicator, the composition comprising:
a lamellar particulate material in a proportion by weight that is greater than or equal to 5% by weight relative to the total weight of the composition; and
a hydrocarbon, silicone, and/or fluorinated binder present in a range of 0.1% to 7% by weight of the total composition.

In embodiments, the method of applying makeup and/or a skincare composition of the invention is for smoothing skin-relief imperfections.

The use of a vibrating applicator associated with loose powder, as defined above, unexpectedly makes it possible to obtain a makeup and/or skincare result that is smoother, more uniform, and less powdery than with applicators known in the art.

The result is particularly visible on women that have skin-relief imperfections such as dilated pores.

In embodiments, the use of a vibrating applicator makes it possible to reduce the tendency of the powder to blow about during application, thereby decreasing any risk of soiling.

Applying vibration to the skin further makes it possible to massage the epidermis with a view to obtaining a sensation of well-being in the application zone. Applying said vibration may induce a biological response in the cells of the epidermis and/or of the dermis, by stimulating mechanoreceptors (e.g. integrins), thereby making it possible to improve the thickness of the skin and/or to improve the radiance of the complexion and/or to improve the mechanical properties of the skin (firmness, elasticity, tonicity).

Vibrating applicators are known.

Patent FR 1 223 254 describes a method of distributing makeup compositions on the skin by means of a vibrating applicator.

Applications FR 2 882 506 and WO 2006/090343 describe different types of vibrator applicator for applying makeup compositions.

Application EP 1 842 520 describes a vibrator applicator that includes a damper that attenuates the transmission of vibration to the user's hand.

Application FR 2 904 923 describes a vibrator device and a method of applying makeup using such a device, the device including fastener means for removably fastening to a finger. That application mentions the possibility of applying a loose powder.

In exemplary embodiments of the present invention, the powder may be applied to the applicator without subjecting the applicator to vibration, by bringing the applicator into contact with the composition contained in a container. The applicator may then be subjected to vibration after applying the composition, while the applicator is still above the container, so as to eliminate any surplus of composition on the applicator, the surplus falling back into the container. In a variant, the applicator may include an internal supply of composition that is delivered during application.

In embodiments, the powder may be applied to bare or madeup skin.

Other exemplary embodiments include a cosmetic kit comprising:
a container containing a cosmetic composition in the form of a loose powder:
a lamellar particulate material in a proportion by weight that is greater than or equal to 5% relative to the total weight of the composition;
a hydrocarbon, silicone, and/or fluorinated binder; powder;
an applicator; and
a vibration source for subjecting the applicator to vibration at least when the composition is applied to the skin and/or when the composition is applied to the applicator.
a hydrocarbon, silicone, and/or fluorinated binder.

The applicator may, for example, comprise a foam, flocking, a brush, or an optionally-woven fabric, as an application surface for coming into contact with the skin.

In embodiments, the applicator may be housed in the container when said container is closed.

### Vibration Source

In embodiments, the application surface is subjected to vibration, the vibration coming from a vibration source.

A suitable vibration source produces vibration that can be obtained in various ways, in particular mechanically, electronically, or electromechanically, and the like.

In embodiments, the packaging and applicator device shown in FIG. 1, and described below, includes a vibration source that enables a vibration to be produced when taking and applying composition, the vibration being produced on an application surface that comes into contact with keratinous material while it is in use.

In general, the frequency of the vibration may lie in the range 5 hertz (Hz) to 10 kilohertz (kHz), such as in the range 100 Hz to 5000 Hz. In exemplary embodiments, the vibration frequency lies in the range 100 Hz to 1000 Hz, such as in the range 100 Hz to 300 Hz.

The vibration source may comprise, for example, a vibrator comprising a motor and a flyweight that is rotated by the motor and that has its center of gravity located eccentrically relative to the axis of rotation. The motor may be powered electrically by an energy source such as, for example, a cylindrical battery electrically connected to the motor via a switch.

In embodiments, the vibration source may include a vibrator other than an electric motor rotating a flyweight. The vibration source may in particular comprise any electromechanical, piezoelectric, pneumatic, hydraulic, mechanical, electronic, or electromechanical system capable of producing vibration.

In embodiments, the vibration source may comprise a vibration control other than a simple on/off switch, and in particular it may comprise mechanical or electronic control that enables the amplitude and/or the frequency of the vibration to be adjusted. For example, the control may include a potentiometer or a rotary or linear switch enabling at least two speeds of rotation of the electric motor to be selected, when the vibrator includes such a motor.

The vibration source may also comprise a plurality of vibrators, as for example, two vibrators arranged to produce oscillations in different directions. Under such circumstances, the applicator may also, for example, include a selector for selecting the vibrator(s) that is/are intended to be put into operation.

Where appropriate, the vibration source may be oriented by the user so as to cause the application element defining the vibrating application surface to vibrate with a vibration of a desired orientation.

In embodiments, the vibration source may comprise an energy source other than a battery, such as one or more batteries, rechargeable batteries or capacitors. The vibration source may be arranged in such a manner as to be suitable for being recharged with electricity when it is placed on a base. Where appropriate, the vibration source may be powered, optionally, via a transformer.

In embodiments, the vibration source may be mounted in multiple ways in a corresponding housing of the applicator or the packaging and dispenser device. The manner in which the vibration source is mounted may be designed to encourage vibration to be transferred towards the application surface or towards the grip surface, for example.

By way of example, the vibration source may be placed in the applicator with a resilient damper interposed between the housing of the applicator and the vibration source. The damper may, for example, comprise an elastomer gasket.

In embodiments, the vibration source may also be carried on a user's finger, as described in application FR 2 904 923.

### BRIEF DESCRIPTION OF THE DRAWINGS

FIG. 1 is an illustration of a cosmetic kit according to a first embodiment.

FIG. 2 is an illustration of a cosmetic kit according to a second embodiment.

FIG. 3 is an illustration of a cosmetic kit according to a third embodiment.

### EMBODIMENTS

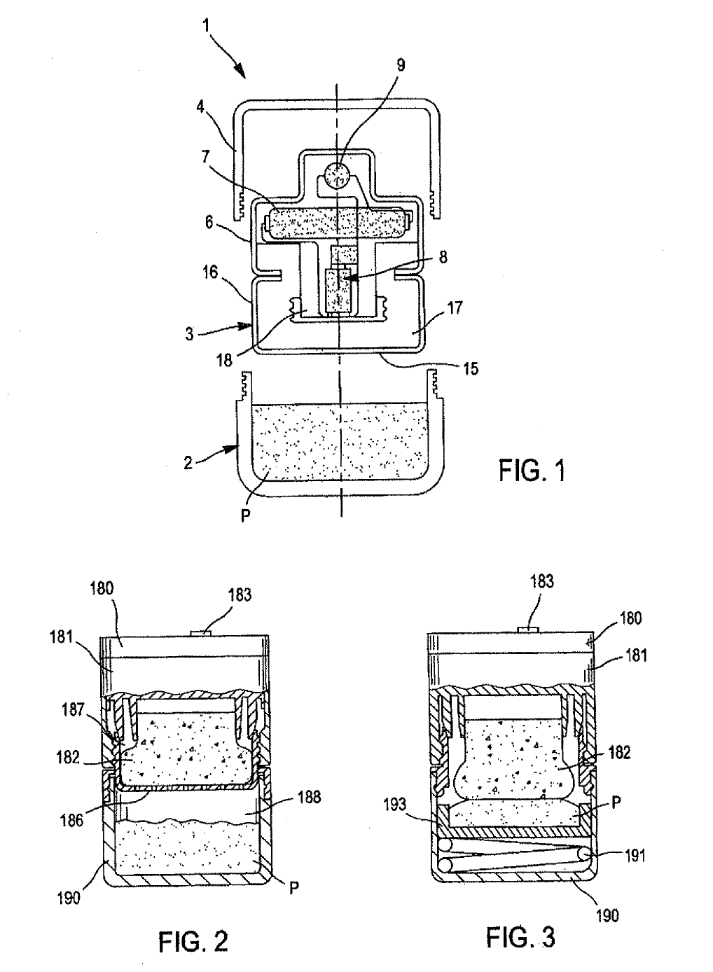

The kit 1 shown in FIG. 1 comprises a container 2 containing the loose powder P, and an applicator 3 capable of being housed inside the container when it is closed by a closure cap 4. The applicator 3 has a grip portion 6 housing the vibration source 8 that includes an energy source 7 and an on/off switch 9. In this example, the vibration source 8 comprises an electric motor rotating a flyweight, however the vibration source could be made in any other way. The application surface 15 of the applicator 3 is defined, for example, by a skin 16 that may cover foam 17, as shown. The motor and the flyweight are contained in a housing 18 of the applicator.

As illustrated in. FIG. 2, the container 2 may optionally include a perforated wall 186 that separates the compartment containing the loose powder P from the compartment 188 for applying the composition to the application surface 187, and as further described in application WO 2006/090343, the content of which is incorporated in its entirety herein by reference.

In the variant embodiment shown in FIG. 2, the device includes a vibration source 180 that can optionally be fastened in removable manner onto an applicator 181 carrying an applicator element 182 comprises a foam, for example.

The vibration source can be switched on by a switch 183 that is provided on an end face of the applicator.

In the embodiment illustrated in FIG. 3, the applicator element 182 is loaded with the composition P through a perforated wall 186 that separates a housing 187 for receiving the applicator element when the container is closed by the applicator, from a space 188 containing the supply of composition. The applicator is fastened onto the container 190 by screw-fastening, for example.

In the example illustrated in FIG. 3, the composition P is contained in a cup 193 that is housed in a container 190 onto which the applicator 181 can be fastened.

By way of example, the applicator has an applicator element 182 that engages inside the container when the applicator is in place thereon. In embodiments, the applicator may comprise a foam.

The applicator element 182 comes into contact with the composition P present in the cup 193, the cup being pressed against the applicator element 182 by a resilient return member, e.g. a spring 191 that is interposed between the cup 193 and the bottom end of the container 190. When the vibration source 180 is put into operation, e.g. by pressing on the switch 183, the vibration that is transmitted to the applicator element 182 serves to disperse the composition P and to load the applicator element 182.

The applicator may comprise any applicator element, e.g. a brush, a paint brush, a flocked tip, a sintered element, a wipe, or the like.

In another variant not shown, the applicator includes the container containing the powder to be applied and a vibration source that is secured to the container while in use. By way of example, the container includes a neck on which there is mounted a support piece for supporting an applicator member, such as a foam that is sufficiently porous to enable the powder to pass therethrough. In a variant that is not shown, the vibration source forms part of a removable unit that enables it to be removed and/or reused with another container.

The applicator may include any applicator element, e.g. a brush, a paint brush, a flocked tip, a sintered piece, or a wipe. In a variant that is not shown, a vibration source of the kind described in application FR 2 904 923, the disclosure of which is incorporated herein by reference in its entirety, is mounted on a user's finger. The application surface may be defined by the end of the finger that comes into contact with the composition that is to be taken and applied, or in a variant by an applicator pad that is fastened on the finger.

### Loose Powder

### Binder

A loose powder preferably contains at least one binder. The binder may comprise hydrocarbon and/or silicone and/or fluorinated compounds.

The term “binder” means an oily phase that may include oil and/or a pasty compound and/or a waxy compound.

The binder compounds may be hydrocarbon and/or silicone and/or fluorinated compounds.

In exemplary embodiments, the binder includes at least one silicone derivative. The term “silicone derivative” encompasses all compounds containing silicone and belonging to one of the following families: silicone oils; silicone resins; silicone waxes; silicone gums; and silicone elastomers.

Examples of silicones of each of the families are described in application FR 2 881 644, the disclosure of which is incorporated herein by reference in its entirety.

In embodiments, the binder may contain a silicone content lying in the range 10% to 100% by weight of total composition.

Examples of suitable hydrocarbon compounds are:
hydrocarbon oils of animal origin, such as perhydrosqualene;
hydrocarbon-containing vegetable oils such as liquid fatty acid triglycerides containing 4 to 10 carbon atoms, for example heptanoic or octanoic acid triglycerides, or sunflower, corn, soybean, squash, grapeseed, sesame seed, hazelnut, apricot kernel, macadamia nut, castor, or avocado oil, caprylic/capric acid triglycerides such as those sold by Stearineries Dubois or those sold under the trade names MIGLYOL® 810, 812, and 818 by Dynamit Nobel, jojoba oil, and shea butter;
linear or branched hydrocarbons of mineral or synthetic origin, such as VASELINE®, polydecenes, and hydrogenated polyisobutene such as PARLEAM®;
synthesized esters and ethers such as R₁COOR₂ formula oils in which R₁ represents a higher fatty acid residue having 6 to 29 carbon atoms, and R₂ represents a hydrocarbon chain having 3 to 20 carbon atoms, e.g. PURCELLIN® oil, isopropyl myristate, 2-ethylhexyl palmitate, 2-octyldodecyl stearate, 2-octyldodecyl erucate, isostearyl isostearate, arachidyl propionate, 2-octyldodecyle benzoate; hydroxylated esters such as isostearyl lactate, octylhydroxystearate, octyldodecyl hydroxystearate, diisostearylmalate, triisocetyl citrate; polyol esters such as propylene glycol dioctanoate, neopentylglycol diheptanoate, diethyleneglycol diisononanoate; and pentaerythritol esters;
fatty alcohols that are liquid at room temperature, with a branched and/or unsaturated carbon-based chain containing 12 to 24 carbon atoms, such as octyldodecanol, 2-hexyldecanol, 2-butyloctanol, 2-undecylpentadecanol, or oleic isostearyl alcohol.

Examples of suitable silicone compounds include:
Linear polysiloxanes having a degree of polymerization that is preferably in the range about 6 to 2000. Examples include: polydimethylsiloxanes (PDMS) of viscosity that is greater than 10 pascals per second (mPa.s); phenyldimethicones; phenyltrimethicones; polyphenylmethyl-siloxanes; and mixtures thereof.
silicone waxes such as substituted linear polysiloxanes. Examples of suitable silicone waxes include: polyether silicone waxes; alkyl or alkoxy-dimethicones having 16 to 45 carbon atoms;
silicone gums such as polysiloxanes of high molecular weight of about 200,000 to 1,000,000 and having a viscosity that is greater than 500,000 mPa.s. Silicone gums may be used alone or mixed with a solvent such as a polydimethylsiloxane or polyphenylsiloxane oil;
silicone resins having the formula: [(RR′R″)3SiO1/2]×[SiO4/2]y in which R, R′, and R″ independently represent a branched or linear alkyl chain of 1 to 10 carbon atoms, or a phenyl radical, and x and y are such that the ratio (RR′R″)3SiO1/2/SiO4/2 lies in the range 0.5/1 to 1.5/1; said silicone resins may be used alone or conveyed with a solvent such as cyclic silicones;
phenylated silicones such as: phenyltrimethicones; diphenyldimethicones; phenyldimethicones; diphenyl methyldiphenyl trisiloxanes; and phenyl trimethylsiloxy diphenylsiloxanes.

Examples of fluorinated compounds include:
fluorinated silicones; and perfluoropolyethers.

In particular, in exemplary embodiments of the present disclosure, the binder is a silicone compound.

The loose powders comprise a binder content lying in the range 0.1% to 7% by weight relative to the total weight of the composition, such as in the range 1% to 6%, or in the range 2% to 5%.

### Lamellar Particulate Material

The advantage of the lamellar shape is to promote spreading of the powder with the vibrating applicator.

In addition to the binder, the loose powder comprises a powder phase that is made up of lamellar particulate materials, in part.

The term “lamellar particulate material” means a particulate material having one dimension that is much smaller than the other two. Such lamellar particulate materials most often have a thickness E that is much less than their length L1 or their width L2.

Preferably, the ratio E/L1 and E/L2 is less than or equal to 0.5, such as less than or equal to 0.3, or less than or equal to 0.1.

The mean thickness E of the lamellar particulate materials may lie in the range 0.1 micrometers (gm) to 5 μm, such as in the range 0.2 μm to 3 μm.

The mean length L1 of the lamellar particulate materials may lie in the range 1 μm to 200 μm, such as in the range 2 μm to 70 μm.

The mean width L2 of the lamellar particulate materials may lie in the range 1 μm to 200 μm, such as in the range 2 μm to 70 μm.

The lamellar particulate material used in the invention may be organic or inorganic.

In embodiments, the lamellar particulate material may be colorless, for example as a filler, or it may be colored so as to impart a color effect, for example, as pigments, e.g. nacres.

Examples of lamellar particulate materials include fillers, pigments, in particular inorganic pigments, and nacres. In embodiments, it is possible to use lamellar fillers and/or lamellar nacres.

In embodiments, suitable lamellar fillers include: talc; mica; barium sulfate; kaolin; precipitated calcium carbonate; magnesium carbonate and hydrocarbonate; hydroxyapatite; lauroyl lysine; glass; polytetrafluoroethylene (PTFE) particles (such as CERIDUST® 9205 F by Clariant, or Fluoropure 103 C by Shamrock Technologies); boron nitride (Ceram Blanche 1 and Ceram Blanche by SPCI and PUHP by Saint-Gobain Ceramics); lamellar silica (SG Flake 3M by Maprecos and Chemicelen by Sumitomo); and mixtures thereof.

In embodiments, suitable lamellar nacres include: bismuthoxichloride; mica coated with titanium or with bismuth oxychloride (BIRON® LF 2000 by Merck); bismuth oxychloride and zinc oxide powder (such as PEARL® II UCR by Farmaquimia); and mixtures thereof.

In embodiments, suitable inorganic pigments in lamellar form include: zirconium oxide; aluminum oxide; and mixtures thereof.

In embodiments, other suitable lamellar include: mica coated with titanium, or with bismuth oxychloride, or with iron oxides, or with organic pigment of the above-mentioned type, and nacres based on bismuth oxychloride.

In embodiments, lamellar particulate materials that are used in the provide a refractive index that is greater than or equal to 1.7 and that are suitable for imparting good coverage after application of the composition to the skin. In embodiments, lamellar particulate materials are selected that have a refractive index lying in the range 1.7 to 2.2. Suitable lamellar particulate materials include: boron nitride; bismuth oxychloride; aluminum oxide; zirconium oxide; and mixtures thereof.

In embodiments, the lamellar particulate materials are selected from: bismuth oxychloride; boron nitride; and mixtures thereof.

The lamellar particulate material content is selected as a function of the result desired, and in particular with a view to obtaining a composition that presents good coverage and that is easy to apply in conjunction with the vibrating applicator of the present disclosure.

In embodiments, the lamellar particulate material content in the invention may lie in the range 5% to 90% by weight relative to the total composition weight, 10% to 80% by weight, or 20% to 60% by weight of total composition.

In exemplary embodiments, each of bismuth oxychloride and/or boron nitride is used in a content lying in the range 2% to 50% by weight relative to the total weight of said composition, such as in the range 5%© to 40% by weight of total composition.

### Non-Lamellar Particulate Material

The powder phase of the composition may further comprise non-lamellar particulate materials of organic or inorganic nature of any shape, whatever the crystal form (e.g. spherical, cubic, hexagonal, orthorombic, etc).

The non-lamellar particular materials may be fillers and/or pigments.

In embodiments, suitable non-lamellar fillers include: non-lamellar silica; polyamide (NYLON®), poly-β-alanine and polyethylene powders; starch; hollow polymeric microspheres such as those made of polyvinylidene chloride/acrylonitrile such as EXPANCEL® (Nobel Industrie) and those made of acrylic acid copolymers; silicone resin microbeads (e.g. TOSPEARLS® by Toshiba); elastomeric polyorganosiloxane particles; polyurethane powders; composite fillers; hollow silica microspheres; glass or ceramic microcapsules; metallic soaps derived from carboxylic organic acids having 8 to 22 carbon atoms, preferably 12 to 18 carbon atoms, e.g. zinc, magnesium, or lithium stearate; zinc laurate; and magnesium myristate.

In embodiments, suitable non-lamellar pigments include: titanium dioxide, possibly with surface treatments; oxides of cerium; and oxides of zinc, magnesium, iron (black, yellow, or red), or chromium; manganese violet; ultramarine blue; chromium hydrate; and ferric blue; and metal powders such as aluminum powder and copper powder; and mixtures thereof.

In exemplary embodiments of the present disclosure, in addition to one or more lamellar particulate materials as described above, the composition may comprise at least one non-lamellar particulate material of spherical shape.

In embodiments, the particulate material(s) of spherical shape is/are selected from inorganic pigments and preferably iron oxides; titanium oxide; zinc oxide; and mixtures thereof.

In exemplary embodiments, the proportion by weight of pigments, in particular inorganic pigments, in said composition of the invention is greater than or equal to 1% by weight.

In embodiments where the powder phase further includes non-lamellar particulate materials, the lamellar particulate material content preferably lies in particular in the range 10% to 100% by weight relative to the total weight of said particulate materials, such as in the range 20% to 80% by weight.

In exemplary embodiments, when the powder phase includes lamellar particulate materials and non-lamellar particulate materials, the proportion of one relative to the other lies in the range 60% to 70% of lamellar particulate materials to 30% to 40% of non-lamellar particulate materials by weight, relative to the total weight of said particulate materials.

### Other Coloring Agents

The composition may further include at least one other coloring agent, such as: organic pigments; a material having a specific optical effect; and/or liposoluble colorants.

Suitable organic pigments include: carbon black; D & C type pigments; and lakes, in particular lakes based on cochenille carmine, on barium, strontium, calcium, or aluminum.

In embodiments, suitable material having a specific optical effect include: particles having a metallic glint; goniochromatic coloring agents; diffractive pigments; thermochrome agents; brightening agents; and fibers, in particular, interference fibers.

In embodiments, examples of liposoluble colorants are: Sudan red; DC Red 17; DC Green 6; β-carotene; soya bean oil; Sudan brown; DC Yellow 11; DC Violet 2; DC Orange 5; and quinolene yellow.

In embodiments, additional pigments may be present in a content lying in the range 0.01% to 60% by weight relative to the total weight of the composition, in the range 0.5 to 55% by weight, such as in the range 1% to 45% by weight.

In embodiments, the liposoluble colorants may be present in the composition in a content lying in the range 0.0001% to 5% by weight relative to the total weight of the composition, such as in the range 0.001% to 3% by weight, or in the range 0.01% to 2% by weight of total composition.

### Active Ingredients

The loose powder may further comprise at least one cosmetically or dermatologically active ingredient, or have none present. Suitable cosmetically, dermatologically, hygienically, or pharmaceutically active ingredients include moisturizing agents (polyols such as glycerine), vitamins (e.g. C, A, E, F, B, or PP), essential fatty acids, essential oils, ceramides, sphingolipids, sunscreens that are liposoluble or in the form of small (<1 μm) particles, and specific skin treatment active ingredients (protective agents, antibacterials, anti-wrinkle agents, etc.), self-tanning agents. In embodiments, the active ingredients may be used in concentrations in the range 0 to 10% relative to the total weight of the composition, such as in the range 0.001% to 5%.

In embodiments, the loose powder may also comprise ingredients that are routinely used in cosmetics, such as: thickeners; surfactants agents; oligo-elements; moisturizing agents; softeners; sequestering agents; fragrances; alkalinizing or acidifying agents; preservatives; antioxidants; UV filters; or mixtures thereof.

In embodiments, depending on the envisaged application, the loose powder may comprise constituents that are conventionally used in the fields under consideration, and that are present in quantities appropriate to the desired dosage form.

The skincare composition may be a tinted powder for the face or for the body.

The makeup composition for keratinous material may in particular be a makeup composition for the skin such as: a foundation in particular for application to the face or the neck; an antiwrinkle composition; a complexion corrector; a blusher; a makeup composition for the body.

In exemplary embodiments, it is a makeup composition for the skin, in particular for the skin of the face, such as a foundation.

### EXAMPLES

### Example 1

### Applying a Loose Powder with Vibrating Applicator

### Example of Loose Powder

| | | % by |
|---|---|---|
| | | weight |
| A1 | Yellow iron oxide | 4.00 |
| | Red iron oxide | 1.30 |
| | Black iron oxide | 0.36 |
| | Titanium dioxide | 20.00 |
| | Zinc oxide sold under the reference Z-COTE ® by BASF | 20.00 |
| | Bismuth oxychloride | 30.00 |
| | Boron nitride | 6.00 |
| | Talc³ | 13.74 |
| | Carnauba wax sold under the reference MICROCARE ® | 1.00 |
| | 350 by Micropowders | |
| A2 | Poly methyl cetyl dimethylsiloxane sold under the | 0.37 |
| | reference ABILWAX 9801 by Goldschmidt | |
| | PDMS + Trimethyl siloxy silicate sold under the reference | 0.59 |
| | DC 593 by Dow Corning | |
| | PDMS (10 cSt) | 1.89 |
| | Tri isocetyl citrate | 0.15 |
| | Preservatives | 0.60 |
| | TOTAL | 100% |

¹Bismuth oxychloride sold under the reference BIRON ® LF 2000 by Merck.
²Boron nitride sold under the reference PUHP 3008 by Saint-Gobain Ceramics.
³Talc sold under the reference LUZENAC ® 00 by Luzenac.

### Mode of Operation

All of the ingredients of phase A1 were mixed together in a Lodige mixer for 15 minutes (without emptying).

While stirring, the ingredients of phase A2 were added into A1 and were mixed together for 15 minutes.

The mixture obtained was then ground in a pin grinder at a speed of 1800 revolutions per minute (rpm).

The mixture obtained was screened on a 400 micrometer screen.

### Applying Makeup

The loose powder was taken with an applicator as described in FIG. 1 without vibration, and then the powder was applied to each half of the face.

On the first half of the face, the applicator was used in vibrating mode at 7000 rpm (117 Hz), whereas on the other half, the same applicator was used without vibration.

It was observed that vibration facilitates application, making it more rapid. The makeup effect obtained with vibration is smoother, more uniform, and less powdery.

This positive effect of vibration is particularly visible for women that have skin with dilated pores.

### Example 2

### Evaluation of the Impact of the Shape of the Particulate Materials

Comparative makeup applications were performed on a panel of 6 people that use foundation with two formulae respectively including lamellar particulate materials for formula 1 or spherical particulate materials for formula 2 (comparative).

Said compositions were prepared as described above in Example 1.

| | | Fomula 1 | Formula 2 |
|---|---|---|---|
| Phase | Ingredients | by weight % | by weight % |
| A1 | talc | 14.89 | 14.89 |
| A2 | Yellow iron oxide | 3.47 | 3.47 |
| | Red iron oxide | 1.00 | 1.00 |
| | Black iron oxide | 0.34 | 0.34 |
| A3 | Bismuth oxychloride | 30.00 | |
| | Boron nitride | 6.00 | |
| | Methylsilsesquioxane resin | | 36.00 |
| | microbeads (grain size | | |
| | 4.5 μm) sold under the trade | | |
| | name Tospearl by GE | | |
| | Toshiba silicones | | |
| | Titanium dioxide | 20.00 | 20.00 |
| | Zinc oxide | 20.00 | 20.00 |
| | Carnauba wax sold under the | 1.00 | 1.00 |
| | reference MICROCARE ® | | |
| | 350 by Micropowders | | |
| B | Tri isocetyl citrate | 0.1650 | 0.1650 |
| | PDMS (10 cSt) | 2.0757 | 2.0757 |
| | PDMS + Trimethyl siloxy | 0.6534 | 0.6534 |
| | silicate sold under the | | |
| | reference DC 593 by Dow | | |
| | Corning | | |
| | Cetyl dimethicone | 0.4059 | 0.4059 |
| | | 100.00 | 100.00 |

### Protocol for Applying Makeup

0.06 grams (g) of each formula were applied to half faces of a panel of six models (people that use foundation) by means of a vibrating applicator. Application was free: each model took said composition (formula 1) with an applicator as described in FIG. 1 without vibration, then applied it with vibration to one half of the face. In the same way, each model took the composition (comparative formula 2) with an applicator as described in FIG. 1 without vibration and applied it with vibration to the other half of the face. The application time was not set, so as to enable the models to obtain the best makeup effects for the different formulae; the mean application time was about 1 minute for applying both formulae, each to half-faces.

Each model evaluated the application of the two compositions in terms of uniformity of the makeup, coverage, and matteness.

For each of the evaluated criteria, the six models marked formula 1 (of the invention) more favorably than formula 2:

| Tested formulae | Uniformity | Coverage | Matteness | Powder effect |
|---|---|---|---|---|
| Formula 1 | +++ | ++++ | ++++ | ++ |
| (lamellar | | | | |
| particulate | | | | |
| materials) | | | | |
| Formula 2 | ++ | ++ | +++ | ++++ |
| (spherical | | | | |
| (particles) | | | | |

The results show that on application, the makeup with the lamellar particulate materials (formula 1) was more uniform than the makeup with the spherical particulate materials. In addition, the makeup result, while exhibiting greater coverage and more matte results, further imparted a less powdery effect with respect to formula 1, and was thus smoother.

### Example 3

### Evaluation of the Impact of the Nature of the Binder

The impact of the nature of the binder on applying the makeup by means of a vibrating applicator was evaluated using two compositions both in the form of loose powder and respectively containing a silicone binder (formula 3) or a hycrocarbon binder (formula 4).

| | | Formula 1 | Formula 3 |
|---|---|---|---|
| Phase | INCI name | by weight % | by weight % |
| A1 | Yellow iron oxide | 14.89 | 14.89 |
| A2 | Red iron oxide | 3.47 | 3.47 |
| | Yellow iron oxide | 1.00 | 1.00 |
| | Black iron oxide | 0.34 | 0.34 |
| A3 | Boron nitride | 30.00 | 30.00 |
| | Bismuth oxychloride | 6.00 | 6.00 |
| | Titanium dioxide | 20.00 | 20.00 |
| | Zinc oxide | 20.00 | 20.00 |
| | Carnauba wax sold | 1.00 | 1.00 |
| | under the reference | | |
| | MICROCARE ® 350 | | |
| | by Micropowders | | |
| B | Isocetyl stearate | | 3.30 |
| | Tri isocetyl citrate | 0.1650 | |
| | PDMS (10 cSt) | 2.0757 | |
| | PDMS + Trimethyl | 0.6534 | |
| | siloxy silicate sold | | |
| | under the reference | | |
| | DC 593 by Dow | | |
| | Corning | | |
| | Cetyl dimethicone | 0.4059 | |
| | | 100.00 | 100.00 |

The protocol for applying said compositions to half faces of a panel of six models, and by means of a vibrating applicator, was the same as described in Example 2.

In addition to the criteria of uniformity, coverage, and matteness that were appreciated in equal manner for both compositions (+++), the powder containing the silicone binder applied with the vibrating applicator gave an even better result, in particular it was less powdery and relief was less marked.

All of the results show that the combination of lamellar particulate materials with a silicone binder in the powder formula, and applied with a vibrating applicator, enables a better application and makeup result to be obtained. The application is more uniform, the makeup result is more covering and more matte, but without a powdery effect. In addition, relief is less marked. The coverage result without powdery effect and without marking relief is particularly advantageous, considering the difficulties in obtaining that result by conventional methods.

The term “comprising a” is synonymous with “comprising at least one” and “lying in the range” should be understood as including the bounds.

It will be appreciated that the present disclosure is not limited to the above applicators, compositions, and cosmetic kits.

Variations of the above-disclosed and other features and functions, or alternatives thereof, may be desirably combined, and are intended to be encompassed by the following claims.

1. A method of applying makeup and/or a skincare composition to skin, comprising:
applying a cosmetic composition in the form of a loose powder with a vibrating applicator, the composition comprising:
a lamellar particulate material in a proportion by weight that is greater than or equal to 5% relative to the total weight of the composition; and
a hydrocarbon, silicone, and/or fluorinated binder, in the range of 0.1% to 7% by weight of the total weight of the composition.

2. The method according to claim 1, further comprising applying the powder to the applicator without subjecting the applicator to vibration, by bringing the applicator into contact with the composition contained in a container.

3. The method according to claim 1, further comprising subjecting the applicator to vibration after applying the composition to the applicator, while the applicator is still above the container, so as to eliminate any surplus of composition on the applicator.

4. The method according to claim 1, wherein the composition is applied to bare skin.

5. The method according to claim 1, wherein the lamellar particulate material is selected from the group consisting of fillers, nacres, and mixtures thereof.

6. The method according to claim 1, wherein the lamellar particulate material is selected from the group consisting of talc, mica, barium sulfate, kaolin, precipitated calcium carbonate, magnesium carbonate and hydrocarbonate, hydroxyapatite, polytetrafluoroethylene particles (PTFE), bismuth oxychloride, mica coated with titanium or with bismuth oxychloride, bismuth oxychloride and zinc oxide powder, boron nitride, lamellar silica, aluminum oxide, zirconium oxide, and mixtures thereof.

7. The method according to claim 1, wherein the lamellar particulate material comprises bismuth oxychloride, boron nitride, or mixtures thereof.

8. The method according to claim 1, wherein the composition further comprises one non-lamellar particulate material.

9. The method according to claim 8, wherein the non-lamellar particulate material is selected from pigments.

10. The method according to claim 9, wherein the proportion by weight of pigments is greater than or equal to 1% of the total weight of the composition.

11. The method according to claim 1, wherein the binder comprises hydrocarbon, silicone and/or fluorinated binder selected from the group consisting of hydrocarbon oils of animal origin, hydrocarbon-containing vegetable oils, linear or branched hydrocarbons of mineral or synthetic origin, synthesized esters and ethers, hydroxylated esters, polypol esters, pentaerythritol esters, fatty alcohols that are liquid at room temperature and have a branched and/or unsaturated carbon-based chain containing 12 to 24 carbon atoms, linear polysiloxanes, silicone waxes; polyether silicone waxes, alkyl or alkoxy-dimethicones having 16 to 45 carbon atoms, silicone gums, phenylated silicones, fluorinated silicones, perfluoropolyethers, perfluorinated alkanes, and silicone resins having the formula: [(RR′R″)3SiO1/2]×[SiO4/2]y in which R, R′, and R″ independently represent a branched or linear alkyl chain of 1 to 10 carbon atoms, or a phenyl radical, and x and y are such that the ratio (RR′R″)3SiO/2 to SiO₄/2 lies in the range 0.5/1 to 1.5/1.

12. A cosmetic kit comprising:
a container containing a loose-powder cosmetic,
an applicator; and
a vibration source for subjecting the applicator to vibration at least when the composition is applied to the skin and/or when the composition is applied to the applicator,
wherein the loose-powder cosmetic comprises a lamellar particulate material in a proportion by weight that is greater than or equal to 5% relative to the total weight of the composition; and
at least one binder selected from the group consisting of a hydrocarbon, silicone, and fluorinated binder.

13. The kit according to claim 12, wherein the applicator comprises an application surface selected from the group consisting of a foam, a brush, flocking, and fabric.

14. The kit according to claim 12, wherein the lamellar particulate material comprises bismuth oxychloride and/or boron nitride, the bismuth oxychloride and/or boron nitride content being present in the composition in a range of 5% to 90% by weight of the total composition.

15. The kit according to claim 12, wherein the binder comprises silicone.

16. The kit according to claim 12, wherein the lamellar particulate material comprises bismuth oxychloride and/or boron nitride, the bismuth oxychloride and/or boron nitride content being present in the composition in a range of 20% to 60% by weight of the total composition.

17. The method according to claim 11, wherein the hydrocarbon oils of animal origin comprise perhydrosqualene;
the hydrocarbon-containing vegetable oils comprise liquid fatty acid tri-glycerides containing 4 to 10 carbon atoms;
the linear or branched hydrocarbons of mineral or synthetic origin are selected from the group consisting of polydecenes and hydrogenated polyisobutene;
the synthesized esters and ethers comprise oils having the formula R₁COOR₂ where R₁ represents a higher fatty acid residue having 6 to 29 carbon atoms, and R₂ represents a hydrocarbon chain having 3 to 20 carbon atoms;
the hydroxylated esters are selected from the group consisting of isostearyl lactate, octylhydroxystearate, octyldodecyl hydroxystearate, diisostearylmalate, and triisocetyl citrate;
the polyol esters are selected from the group consisting of propylene glycol dioctanoate, neopentylglycol diheptanoate, and diethyleneglycol diisononanoate;
the fatty alcohols are selected from the group consisting of octyldodecanol, 2-hexyldecanol, 2-butyloctanol, 2-undecylpentadecanol, and oleic isostearyl alcohol;
the linear polysiloxanes have a degree of polymerization that is in the range of about 6 to 2000 and are selected from the group consisting of phenyldimethicones, phenyltrimethicones, and polyphenylmethyl siloxanes;
the silicone waxes are substituted linear polysiloxanes;
the silicone gums are selected from the group consisting of polysiloxanes of high molecular weight of about 200,000 to 1,000,000 and having a viscosity that is greater than 500,000 mPa.s, used alone or mixed with a solvent; and
the phenylated silicones are selected from the group consisting of phenyltrimethicones, diphenyldimethicones, phenyldimethicones, diphenyl methyldiphenyl trisiloxanes, and phenyl trimethylsiloxy diphenylsiloxanes.

18. The method according to claim 17, wherein the synthesized esters and ethers are selected from the group consisting of isopropyl myristate, 2-ethylhexyl palmitate, 2-octyldodecyl stearate, 2-octyldodecyl erucate, isostearyl isostearate, arachidyl propionate, and 2-octyldodecyle benzoate.

19. The method according to claim 11, wherein the hydrocarbon-containing vegetable oils are selected from the group consisting of heptanoic or octanoic acid triglycerides; sunflower oil; corn oil; soybean oil; squash oil; grapeseed oil; sesame seed oil; hazelnut oil; apricot kernel oil; macadamia nut oil; castor oil; avocado oil; caprylic/capric acid triglycerides; jojoba oil; and shea butter.

20. The method according to claim 17, wherein the linear polysiloxanes are polydimethylsiloxanes having a viscosity that is greater than 10 mPa.s.

21. The kit according to claim 13, wherein the application surface comprises a woven fabric.
